(19) United States
(12) Patent Application Publication
(10) Pub. No.: US 2003/0176511 A1
(43) Pub. Date: Sep. 18, 2003

(54) Compositions and methods for treating cardiovascular disorders

(76) Inventor: Roland Stocker, New South Wales (AU)

Correspondence Address: FISH & RICHARDSON PC, 225 FRANKLIN ST, BOSTON, MA, 02110, US

(21) Appl. No.: 10/332,717

(22) PCT Filed: Jul. 12, 2001
(86) PCT No.: PCT/AU01/00835

(30) Foreign Application Priority Data
Jul. 12, 2000 (AU) PQ 8728

(51) Int. Cl. A61K 31/105; A61K 31/05

(52) U.S. Cl. 514/707; 514/734

(57) ABSTRACT

The present invention relates compositions and methods for the prophylactic or therapeutic treatment of cardiovascular diseases, particularly restenosis and atherosclerosis. These compositions comprise an effective promotor of re-endothelialization, preferably probucol or an analogue thereof, and an inhibitor of lipoprotein oxidation, preferably a probucol-derived bisphenol. Alternatively, the said compositions may comprise a compound that possesses both re-endothelialization-promoting and lipoprotein oxidation-inhibitory effects. This invention further relates to compositions and methods for promoting re-endothelialization.

### TECHNICAL FIELD

The present invention relates to a method of prophylactic or therapeutic treatment of cardiovascular diseases, particularly restenosis and atherosclerosis by a process which both induces re-endothelialization and inhibits the oxidation of lipoproteins. The invention further provides a method of promoting re-endothelialization preferably in a vessel wall.

### BACKGROUND ART

Heart disease can result from many factors relating to poor functioning of heart tissue which may manifest in commonly known conditions such as angina, stroke or heart attack. The underlying mechanisms of heart disease are not completely understood. However, it is known that lipid such as cholesterol are actively involved. These can all contribute to atherosclerosis, i.e., the clogging of arteries, and gradually building deposits that eventually cause heart disease.

Atherosclerosis causes heart attacks, strokes and leads to the death of almost 50% of Australians. The disease involves intimal thickening and the deposition of lipid (primarily derived from low-density lipoprotein, LDL) in the sub-endothelial space. As lesions develop, the elastic lamina separates the intima from the media, allowing proliferating smooth muscle cells to infiltrate the intima and to deposit increased amounts of extra-cellular matrix. A necrotic core (composed of dead cells, lipid deposits and cholesterol crystals) may develop and the disease can also involve the medial layer. Atherosclerosis can develop silently for many years without symptoms. Often serious events are precipitated when a blood clot lodges in the vessel at a site that is already partially blocked as a result of atherosclerosis.

Attempts have been made to understand those factors that trigger a lipid deposit resulting in the narrowing and eventual occlusion of a vessel (stenosis). According to the ‘oxidation theory’ of atherosclerosis, the oxidation of LDL (and other lipoproteins) predominantly occurs in the sub-endothelial space of the vessel wall. Oxidized LDL is pro-atherogenic by promoting the accumulation of lipids in cells, disturbing the normal vasoregulatory function of endothelial cells, being cytotoxic to endothelial and other cells, mediating the generation of a necrotic core, promoting the recruitment of inflammatory cells, and by inducing thrombogenic tissue factor and the expression of adhesion molecules on endothelial cells. Accumulation of lipid by macrophages can induce the secretion of matrix metalloproteinases and cytokines (e.g. IL-8). These thrombotic, adhesive and inflammatory properties of oxidized LDL may be critical for disease progression (whether episodic or continuous) and likely involves episodic damage to the endothelium.

According to the response to injury hypothesis of atherosclerosis, endothelial cell injury, such as can be promoted by oxidized LDL, can itself trigger or contribute to the development of atherosclerosis.

Blocked coronary arteries are commonly treated by balloon angioplasty (BA), a procedure during which a catheter is inserted and a balloon is inflated at the site of stenosis to restore blood flow. Despite excellent acute results, the major limitation of BA is the re-occlusion (or restenosis) of the treated vessel, an event occurring after approximately 40% of the procedures. BA causes damage to the vessel wall including but not restricted to the denudation of the endothelial cell layer. It is thought that excessive neointimal proliferation and extracellular matrix synthesis by modified smooth muscle cells in response to such injury represents an important component of the underlying mechanism of restenosis. In addition, such damage can also cause the recruitment of inflammatory macrophages exacerbating the damage by enhancing the oxidation of lipoproteins and ultimately atherosclerosis. The exposed matrix also has the propensity to react with blood components leading to clots and increasing the incidence of acute myocardial events.

Recent studies referred to in WO 98/42327 (PCT/CA98/00269), suggest geometric remodelling after BA as a concomitant contributor to the process of restenosis. Remodelling is defined as a change in the total arterial or external elastic membrane cross-sectional area (CSA) over time. This geometric remodelling after balloon injury appears to be biphasic, with an initial adaptive increase in arterial CSA vessel (i.e., positive arterial remodelling, reflected by an enlargement of the vessel) followed by a constrictive phase (i.e., negative arterial remodelling) during which the luminal volume of the vessel decreases.

Attempts to modify the restenotic process by pharmacological or mechanical approaches have been disappointing. In particular, statins (lipid-lowering agents with proven anti-atherosclerotic activity) are ineffective in attenuating restenosis. Other agents tested in a controlled manner and yielding negative results include calcium channel blockers, angiotensin-converting enzyme inhibitors, and antithrombotic and antiproliferative agents.

An exception to these negative results is probucol, a lipid-lowering drug with antioxidant properties and used experimentally for the prevention of cardiovascular diseases.

Probucol has the following formula:

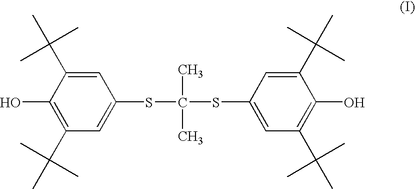

The original use for probucol was in the rubber industry as a polymerization inhibitor. It has since been found to be a hypolipidemic agent as well as an antioxidant. In fact, Probucol has been shown to be effective in preventing restenosis particularly after percutaneous transluminal coronary angioplasty (PTCA). Thus, several small clinical studies listed in PCT/CA98/00269 initially suggested that probucol started four weeks before BA may prevent restenosis. Subsequently, the large randomized MultiVitamins and Probucol (MVP) trial showed that probucol given alone reduced angiographic lumen loss by 68%, restenosis rate per segment by 47% and the need for repeat angioplasty at 6 months by 58% compared to the placebo. However, it is unclear how probucol acts to give these results and whether it acts via inhibition of tissue hyperplasia or improvement in vascular remodelling.

Hence for probucol which is one of the first pharmacological agents shown to prevent coronary restenosis after BA, the mechanism underlying its restenosis-inhibitory effect is not understood. Several activities of probucol are commonly considered. Foremost among these is its antioxidant activity that is thought to attenuate cardiovascular disease by preventing the oxidation of LDL. However, we have obtained evidence that dissociates inhibition of restenosis from the prevention of lipoprotein oxidation.

In addition, it has been suggested that probucol inhibits restenosis by inhibiting smooth muscle cell proliferation (Tanaka et al. Cardiovasc Drugs Ther 1998;12, 19-28). However, this study did not establish whether the observed inhibition of smooth muscle cells by probucol was direct or indirect. Also, direct inhibition of smooth muscle cell proliferation may not always be advantageous as smooth muscle cells provide stability to plaques and thereby reduce the risk of plaque rupture which itself is the key event that causes a clinical event.

Furthermore, it has been suggested that probucol may inhibit restenosis by promoting the proliferation of endothelial cells (Komatsu et al. J Pharmacol Toxicol 1999;41:33-41). This suggestion is based entirely on in vitro studies, the in vivo relevance of which remains unknown. For example, Komatsu et al. used venous endothelial cells that are known to respond differently to arterial endothelial cells which are affected by BA.

However, recovery from BA and prevention of restenosis may involve regaining the integrity of an artery as soon as possible. Probucol has been implicated in preventing restenosis mainly through its reputation as a hypolipodemic agent and an antioxidant which inhibits oxidation of LDL. Apart from its known effect on inhibiting smooth muscle cell proliferation, probucol has not been associated with contributing to the integrity of the arterial wall after BA or similar insult. Therefore it would be desirable to obtain a compound which could initiate the restoration of an intact artery following BA or a similar insult.

Accordingly it is an object of the present invention to overcome or at least alleviate some of the problems of the prior art, or to provide a useful alternative.

### SUMMARY OF THE INVENTION

In a first aspect there is provided a composition comprising an effective promoter of re-endothelialization and an effective inhibitor of lipoprotein oxidation.

Preferably the promoter of re-endothelialization is probucol or an analogue thereof. Preferably the inhibitor of lipoprotein oxidation is a co-antioxidant such as the probucol-derived bisphenol however it will be understood by those skilled in the art that other inhibitors of lipoprotein oxidation can also be used. In that regard the term “effective inhibitor of lipoprotein oxidation”, as used in the context of the present invention, encompasses those inhibitors of lipoprotein oxidation which are effective in vivo in the blood vessel wall. The description provided herein, including cited references, guides those skilled in the art how to identify suitable inhibitors.

Probucol-derived bisphenol has the following structure:

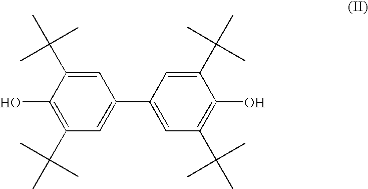

In a second aspect there is provided a composition comprising a novel compound that possesses both re-endothelialization-promoting and lipoprotein oxidation-inhibitory activity.

In a preferred embodiment such novel compound may be a probucol analogue having modifications in the region of the ‘central bridge’ of probucol that allows intra-cellular reduction to a mercaptophenol. For example, the analogue may have the following, or related, structure:

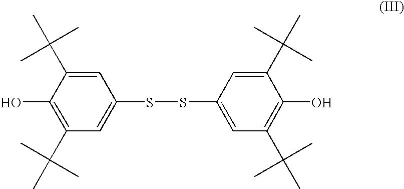

The compound of formula (III) may be formulated on its own or it may be formulated to comprise one or more effective inhibitors of lipoprotein oxidation, for example a co-antioxidant such as bisphenol.

In a third aspect there is provided a method of treating cardiovascular diseases, said method comprising the administration to a subject requiring such treatment an effective promoter of re-endothelialization and an effective inhibitor of lipoprotein oxidation.

Preferably the promoter of re-endothelialization is probucol or an analogue thereof.

Preferably the treatment with the compositions of the present invention promotes re-endothelialization of damaged vessel walls in vivo. Also preferred is a composition which comprises probucol and its bisphenol. Further, the treatment may be prophylactic or therapeutic.

Preferably probucol and the inhibitor are administered simultaneously but it will be understood that they may be administered sequentially in any order to achieve the desired effect.

In a fourth aspect there is provided a method of treating cardiovascular diseases, said method comprising the administration to a subject requiring such treatment a novel compound that possesses both re-endothelialization-promoting and effective lipoprotein oxidation-inhibitory activity.

The preferred compound is the compound of formula (III) as set out above. The compound of formula (III) may be administered in conjunction with another compound which has lipoprotein oxidation-inhibitory activity, for example a co-antixodant such as bisphenol.

In a fifth aspect there is provided a method of treatment which promotes the re-endothelialization of damaged vessel walls in vivo, said method comprising administering to a subject requiring such treatment an effective amount of a promoter of re-endothelialization and an effective inhibitor of lipoprotein oxidation.

Preferably the promoter of re-endothelialization is probucol or an analogue thereof. Even more preferred is the administration of a compound of formula (III) as set out above.

The method of promoting re-endothelialization may also extend to methods of treating conditions associated with endothelial dysfunction, for instance in the control of vascular tone via endothelium-dependent relaxing factor (i.e., nitric oxide produced by eNOS), the deposition of matrix by, and proliferation of, smooth muscle cells, the infiltration of the vessel wall by inflammatory blood cells, and the control of coagulation and platelet aggregation.

In a sixth aspect there is provided a method of treating cardiovascular diseases, said method comprising administering to a subject requiring such treatment an effective amount of probucol or an analogue thereof, to promote re-endothelialization.

In a seventh aspect there is provided a re-endothelialization composition comprising probucol or an analogue thereof and a pharmaceutically accepted carrier.

In an eighth aspect there is provided a re-endothelialization composition comprising a compound of formula (III).

The re-endothelialization composition may further comprise, or may be used in conjunction with, one or more effective inhibitors of lipoprotein oxidation, for example a co-antixodant such as bisphenol.

Unless the context clearly requires otherwise, throughout the description and the claims, the words ‘comprise’, ‘comprising’, and the like are to be construed in an inclusive as opposed to an exclusive or exhaustive sense; that is to say, in the sense of “including, but not limited to”.

### BRIEF DESCRIPTION OF THE FIGURES

FIG. 1 shows cross-sections through the aortic root (panels a and e), aortic arch (panels b and f), descending thoracic aorta (panels c and g) and the proximal abdominal aorta (panels d and h). Sections from controls and probucol-treated animals are shown in panels a to d and e to h, respectively. Sections were taken in close proximity to branching vessels, indicated by arrows. Aortic lesions were clearly smaller in probucol-treated than control mice, except in the case in the aortic root. Note that In response to the large mass of atherosclerosis in the aortic root in the probucol treated mice, the entire root has grown markedly compared to the control (see panel a versus e). In many mice, probucol almost completely abolished lesion formation in the aorta (e.g. panel g).

FIG. 2 shows the plasma lipoprotein profile of apolipoprotein E gene knock out (apoE−/−) mice fed a high fat diet without and with 1% probucol for 24 weeks. Plasma was collected from individual mice, pooled, diluted 1:10 with buffer used for FPLC and 300 μL subjected to size exclusion chromatography. Chromatograms shown are representative of two analyses of independent pooled plasma samples. The horizontal bars indicate the corresponding fractions collected for each lipoprotein pool.

FIG. 3 shows that probucol decreases the ex vivo oxidizability of plasma lipids obtained from apoE−/− mice fed a high fat diet. Pooled plasma obtained from control (open symbols) or probucol-treated (closed symbols) mice was exposed to 5 mM of the aqueous peroxyl radical generator AAPH and incubated under air at 37° C. At the times indicated, aliquots of the reaction mixture were removed and analysed for A, ascorbate (diamonds); B, ubiquinol-9 plus ubiquinol-10 (inverted triangles); C, α-tocopherol (TOH, squares), probucol (cross-hatched squares) and bisphenol (filled triangles); and D, hydroperoxides and hydroxides of cholesteryl esters (CE-O(O)H, circles). Data shown are mean±SD of a single oxidation experiment performed in triplicate using pooled plasma. Where error bars are not shown, error is smaller than the symbol.

FIG. 4 shows re-endothelialization by probucol in cholesterol-fed rabbits after balloon injury. TAA and AA refer to thoracic and abdominal aorta, respectively.

FIG. 5 shows resistance of plasma from apolipoprotein E and LDL receptor gene double knockout (apoE−/−;LDLr−/−) mice receiving bisphenol to lipid peroxidation initiated by aqueous peroxyl radicals. Pooled plasma obtained from at least 3 mice receiving control diet (circles) or diet supplemented with bisphenol (squares) were treated with 5 mM AAPH and incubated at 37° C. At the times indicted aliquots were removed and analysed for α-TOH (A) and CE-O(O)H (B). 100% α-TOH levels correspond to 28±3 and 20±2 μM for control and H 212/43 plasma, respectively. Data represents the mean±SD (n=4 independent experiments).

FIG. 6 shows inhibition of aortic lipoprotein lipid peroxidation in apoE−/−;LDLr−/− mice receiving bisphenol. Pooled aortas from 7-8 mice obtained from control or bisphenol-treated mice were homogenized, the lipid extracted and subjected to HPLC with post-column chemiluminescence detection for analysis of cholesteryl ester hydroperoxides (CE-OOH). Chromatograms correspond to aortas of age-matched control (a or b) or drug-treated mice with low or high plasma levels of bisphenol (c and d, respectively). Under the conditions used, CE-OOH eluted between 8 and 9.5 min. The chemiluminescence negative peaks between 4 and 5.5 min correspond to the elution of tocopherols.

FIG. 7 shows inhibition of atherosclerosis in apoE−/−;LDLr−/− mice receiving bisphenol. Lesion formation was assessed by changes to intima volume when compared with young and age-matched controls receiving standard chow for 8 and 22 weeks, respectively. *Denotes a statistically significant difference with α<0.025 (Wilcoxon test).

FIG. 8 shows inhibition of restenosis in rabbits receiving probucol dithio analog PDA474. The intima and media ratio (IMR) was determined by tracing the intimal and medial areas, and dividing their respective total pixel numbers. Higher IMR indicate larger lesions. *Denotes a statistically significant difference with p<0.05.

### DESCRIPTION OF THE PREFERRED EMBODIMENTS

In one aspect the present invention provides a composition comprising probucol or an analogue thereof and an effective inhibitor of lipoprotein oxidation.

The effective inhibitors of lipoprotein oxidation for the purposes of the present invention are those which are effective in blood vessel walls in vivo. Their identity may be ascertained routinely by in vivo analysis of the effects of the inhibitors in blood vessel walls using a suitable animal model such as Watanabe Heritable Hyperlipidemic (WHHL) rabbits, apoE−/− mice, or cholesterol-fed ballooned New Zealand White rabbits. Alternatively, they may be identified through in vitro assays which are capable of demonstrating such efficacy, such as for example assays described in J Lipid Research 1996, 37:853-867 which is incorporated herein by reference.

The effective inhibitor of lipoprotein oxidation may be a co-antioxidant as described in for example WO 97/38681, J Lipid Research 1996, 37:853-867; J Biol Chem 1995;270:5756-5763. For the purposes of the present invention, “co-antioxidants” are distinguished from “classic antioxidants”, as described in detail in J Biol Chem 1995;270:5756-5763.

While the present invention presents that the composition of probucol or an analogue thereof with a lipoprotein oxidation inhibitor such as a co-antioxidant is beneficial, the composition of probucol with classic antioxidants has been shown to nullify the restenosis-inhibitory activity of probucol (N Engl J Med 1997;337:365-372). Probucol itself is not a co-antioxidant (J Biol Chem 1995;270:5756-5763; J Lipid Res 1996;37:853-867). Suitable co-antioxidants include, but are not limited to, the compounds listed in WO 97/38681; J Biol Chem 1995;270:5756-5763; J Lipid Res 1996;37:853-867.

In another aspect the present invention provides a composition comprising a novel compound that possesses both re-endothelialization-promoting and lipoprotein oxidation-inhibitory activity. Such novel compound may be a probucol analogue having modifications in the region of the ‘central bridge’ to a disulfide. This may allow intra-cellular reduction of the novel compound to a mercaptophenol. For example, the analogue may have the following, or related, structure:

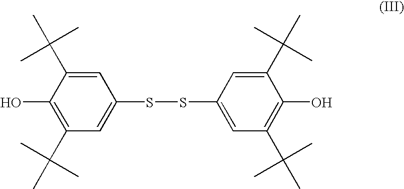

The compound may be prepared by standard synthesis schemes known to the skilled addressee. The substituent designated

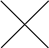

on each of the phenolic rings is intended to represent a t-butyl group.

In yet another aspect the present invention provides a method of treating cardiovascular diseases, said method comprising the administration to a subject requiring such treatment an effective amount of probucol or an analogue thereof and an effective inhibitor of lipoprotein oxidation.

The probucol and inhibitor may be administered simultaneously or sequentially in any order.

The treatment with the compositions of the present invention promotes re-endothelialization of damaged vessel walls in vivo. An effective combination is a composition which comprises probucol and its bisphenol as the lipoprotein oxidation inhibitor however other inhibitors identified by methodologies described or referenced herein could also be used. Further, the treatment may be prophylactic or therapeutic.

In yet another aspect the present invention provides a method of treatment which promotes the re-endothelialization of damaged vessel walls in vivo, said method comprising administering to a subject requiring such treatment an effective amount of probucol or an analogue thereof.

Re-endothelialization is the process whereby an intact endothelial cell layer grows back over a previously denuded (i.e., de-endothelialized) area of the blood vessel. Commonly, the re-growth of endothelial cells is initiated at branching points of smaller vessels and cell growth then progresses into the 25 larger vessel. Re-endothelialization is not identical to the process of endothelial cell proliferation. The former is limited to previously damaged areas, whereas endothelial cell proliferation is a more general process required, for instance in angiogenesis which itself can promote rather than inhibit atherosclerosis (Circulation 1999;99:1726-1732). 30 Re-endothelialization is particularly important for the prevention of restenosis after BA (where the endothelial cell layer of large areas of vessels become removed). For example, the local delivery of vascular endothelial growth factor (a growth factor that specifically promotes the growth of endothelial cells) accelerates re-endothelialization and attenuates intimal hyperplasia in the balloon-injured rat carotid artery (Circulation 1995;91:2793-2801). Re-endothelialization may also be important in atherosclerosis where injury to endothelial cells occurs, for example as a result of the accumulation and toxic properties of oxidized LDL.

The endothelium is a cell layer that lines internal body surfaces such as in the heart, blood and lymphatic vessels and other fluid filled cavities and glands. Endothelium must be induced to re-grow if the integrity of the surface is to be maintained. The integrity of endothelium in blood vessels is of central importance to vascular homeostasis in general and processes related to restenosis and atherosclerosis in particular. The latter include, but are not limited to, the control of vascular tone via endothelium-dependent relaxing factor (i.e., nitric oxide produced by eNOS), the deposition of matrix by, and proliferation of, smooth muscle cells, the infiltration of the vessel wall by inflammatory blood cells, and the control of coagulation and platelet aggregation. Smooth muscle cell proliferation is often implicated in restenosis. Prevention of the proliferation has been effective in inhibiting the progress of restenosis. However, the direct general prevention of smooth muscle cell proliferation may not always be beneficial, as for instance it can decrease the stability of plaques and thereby promote clinical events by promoting plaque rupture.

A method that promotes re-endothelialization may have many advantageous outcomes that contribute to maintaining the integrity of vessel walls. The result can manifest in better circulation, and general well being.

The method of promoting re-endothelialization may also extend to methods of treating conditions associated with endothelial dysfunction for instance in the control of vascular tone via endothelium-dependent relaxing factor (i.e., nitric oxide produced by eNOS), the deposition of matrix by, and proliferation of, smooth muscle cells, the infiltration of the vessel wall by inflammatory blood cells, and the control of coagulation and platelet aggregation.

The promotion of re-endothelialization by administration of probucol may be conducted at any time. For instance re-endothelialization may be promoted before or after angioplasty, PTCA or BA. Preferably, the promotion of re-endothelialization occurs after denudation (removal of endothelial cells). However, the administration of probucol may be made prior to denudation. Preferably, the administration is made prior to a denudation event such as BA. More preferably the probucol is administered 3 to 4 days prior to the denudation event.

The term “vessels” as used herein includes all fluid or air filled vessels of the body which are lined with endothelium. Preferably the vessels are blood vessels. More preferably they are arteries. Arteries are most likely to be blocked by atherosclerotic plaques requiring angioplasty to remove the affecting plaque thereby denuding the endothelial layer.

Damage to vessel walls may occur by any means that strip the vessel of the endothelium preferably it is an arterial injury. The damage may be caused by angioplasty, PTCA, or BA.

The method according to the invention comprises the administration of probucol or an analogue thereof. The term “analogue thereof” includes molecules acting in a similar manner to probucol and having a similar structure to probucol.

The term “effective amount” is used herein to describe an amount effective to promote re-endothelialization in a damaged vessel. The probucol may be administered at an amount which provides approximately 1% of probucol in the diet. Alternatively, probucol may be administered at approximately 500 mg twice daily prior to arterial injury.

Methods of administering probucol will depend on the site where re-endothelialization is to be promoted. Administration may be by the oral, intravenous, intramuscular, subcutaneous, intranasal, intradermal or by suppository routes. Depending on the route of administration probucol may be administered on its own or in combination with one or more active molecules to facilitate the delivery of probucol to a site of action. For instance, liposomes, glycerol, polyethylene glycols, mixtures of oils or buffers, edible carriers preventing or for tablets, capsules may be used.

In another aspect of the present invention, there is provided a method of treating heart disease, said method including administering an effective amount of probucol or an analogue thereof to a patient to promote re-endothelialization.

Heart disease may be any condition of the heart which is associated with vessel function. For instance, loss of integrity of the vessels can lead to poor circulation, atherosclerotic plaque formation, restenosis, angina, stroke or heart attacks. Hence, treatment of heart disease extends to treatment of any of these conditions. Preferably, the heart disease is atherosclerosis.

Although not wishing to limit the present invention to any one hypothesis as to mode of action, it is possible that probucol acts by selectively promoting the re-growth of endothelial cells at areas of the vessel wall that previously have been denuded as a result of angioplasty, PTCA or BA, atherosclerosis, or other arterial injury.

The term “treating” is used herein in its broadest sense to include prophylactic (ie. preventative) treatment as well as treatments designed to ameliorate the effects of heart disease, preferably atherosclerosis. The treatment by use of probucol is aimed at promoting re-endothelialization. It is thought that by doing so, the integrity of the vessel walls is maintained in a healthy state.

In yet another aspect of the present invention there is provided a re-endothelialization composition comprising probucol and a pharmaceutically accepted carrier.

The carrier may be any carrier that is physiologically acceptable to the body. It may be saline, a buffered saline solution or water, or a compound which facilitates delivery of probucol to a site that requires re-endothelialization.

The present invention will now be more fully described with reference to the following examples. It should be understood, however, that the description following is illustrative only and should not be taken in any way as a restriction on the generality of the invention described above.

### EXAMPLES

### Example 1

Ability of Probucol to Inhibit Atherosclerosis in Apolipoprotein E Gene Knock-Out Mice without Inhibition of Lipoprotein Oxidation in the Vessel Wall.

(a) Animals and Diet

Male C57BL/6J mice, homozygous for the disrupted apoE gene (apoE−/−) were fed standard chow (Lab-Feed, Sydney, Australia) until aged 10 weeks. Subsequently mice were fed ad libitum a high fat diet containing 21.2 and 0.15% (wt/wt) fat and cholesterol, respectively with or without 1% probucol (wt/wt). The high fat diet (control and probucol-supplemented) was prepared, according to the specifications of the Harlan Teklad diet TD88137. Control chow did not contain detectable lipid hydroperoxides.

(b) Plasma Oxidation and Lipoproteins

Plasma was obtained from control and probucol mice and aliquots frozen for subsequent determination of lipids. Separate aliquots were acidified with metaphosphoric acid (5%) to stabilize vitamin C prior to freezing and storage at −80° C. The remainder was pooled appropriately and used for ex vivo oxidation initiated by the peroxyl radical generator AAPH and lipoprotein separation by FPLC with UV₂₈₀ₙₘ detection, as described in J Lipid Res. 1 999;40:1104-1112.

(c) Analyses of Lipids and Antioxidants

Lipid-soluble antioxidants and lipids were quantified by HPLC as described in FASEB J. 1999;13:667-675. For ascorbate analysis, frozen acidified samples were thawed, diluted with DPBS to adjust the pH to 7.4 and then immediately subjected to HPLC. Plasma triglycerides were determined enzymatically (Boehringer, Mannheim, Germany).

(d) Removal of Aortas

After bleeding, mice were gravity-perfused for 5 min with DPBS containing 20 μM BHT and 1 mM EDTA (Buffer A) and aortas removed as described in Letters JM, et al (1999). Briefly, hearts, ascending and descending aortas (past the femoral junction) were excised and carefully cleaned. Aortas designated for histology (n=10 and 9 for control and probucol groups, respectively) were perfusion fixed with Buffer A containing 4% (v/v) formaldehyde, transferred (with the hearts attached) into formalin. For biochemistry, aortas (n=22-24 for control and probucol groups) were not fixed as adventitious oxidation takes place using standard fixation procedures. Once cleaned, aortas were separated from the heart taking care to include all aortic material while avoiding heart tissue. To obtain sufficient material for HPLC analysis (i.e., 30-40 mg wet weight tissue) it was necessary to pool 7-8 aortas. Separate pools of aortas were prepared for both groups and then immediately frozen in Buffer A and stored at −80° C. until analyses.

(e) Biochemistry of Aortic Homogenates

Pooled aortas were snap frozen in liquid nitrogen, pulverised, resuspended in Buffer A, homogenised and then either treated with metaphosphoric acid (for ascorbate) or extracted and the hexane fraction analysed for lipid-soluble antioxidants, cholesterol (C), choleseteryl esters (CE), lipid hydroperoxides (LOOH) and cholesteryl ester hydroxides (CE-OH) by HPLC as described in Methods Enzymol. 1994;233:469489. LOOH and CE-OH were measured as markers of lipoprotein lipid oxidation, as they are the primary and major oxidation products formed when lipoproteins from apoE−/− mice undergo oxidation. Bisphenol, probucol and diphenoquinone were analysed by gradient RP-HPLC with compounds eluting at ˜9, 17 and 27 min, respectively. All compounds were quantified by peak area comparison with authentic standards, and protein determined.

(f) Morphometry

Lesions were assessed at four different sites along the aorta, in the aortic root, in the aortic arch, the descending thoracic and in the proximal abdominal aorta, and cross-sections (2-3 μm thick) were prepared and stained with Weigert's hematoxylin-van Gieson.

This example establishes the dissociation of anti-atherosclerotic activity of Probucol from its putative action as an antioxidant that inhibits LDL oxidation as described below.

(a) Aortic Morphometry

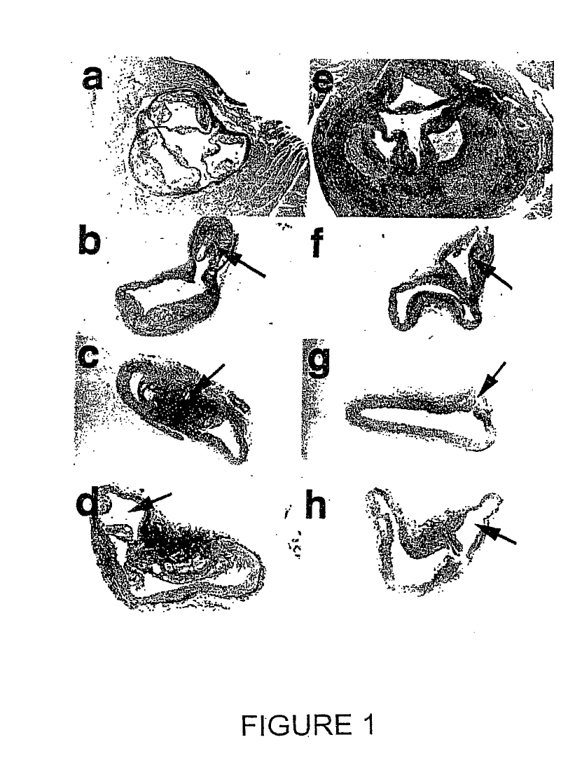

Representative cross-sections from control and probucol-treated groups are shown in FIG. 1. Lesions were found at all sites and covered large areas of the vessel, with the exception of the descending thoracic aorta where lesions were smaller and located around the ostiae of the branching intercostal arteries. The lesion morphology was grossly similar in all regions, with necrotic cores containing cholesterol crystals observed regularly at all sites. Table 1 summarises the lesion sizes observed.

TABLE 1
Site-specific anti-atherogenic effect of probucol on the formation of
atherosclerotic lesions in apoE−/− mice measured as lesion cross-section
areas (μm² × 10⁻³).

| | | | Descending | |
|---|---|---|---|---|
| | | | Thoracic | Abdominal |
| | Aortic Root | Arch | Aorta | Aorta |
| Controls | 810 ± 20 (10) | 319 ± 26 (10) | 34 ± 10 (10) | 121 ± 31 (9) |
| Probucol | 1180 ± 260 (9) | 140 ± 34 (9)* | 5 ± 4 (8)* | 12 ± 12 (3) |
| Treatment | 146% | 44% | 15% | 10% |
| effect | | | | |

Mice were fed a high fat diet in the absence (controls) or presence of 1% (w/w) probucol for 24 weeks before lesions were assessed at different sites.

Analysis of variance showed that probucol significantly but site-dependently affected lesion size (Table 1), as indicated by a significant interaction term (P=0.001). Direct comparisons showed significantly smaller lesions in the aortic arch and descending thoracic aorta in probucol versus control (Table 1). The few results obtained from the abdominal aorta also indicated an anti-atherogenic effect of probucol. Previous studies reported that probucol enhances lesion formation in the aortic root of female and male apoE−/− mice fed a normal chow. (J Clin Invest. 1997; 99:2858-2866; Circulation. 1999;99:1733-1739). Consistent with this, lesions at this site were larger in probucol than control male mice fed a high fat chow (Table 1), although this difference did not reach statistical significance. Table 1 also shows that the lesion enhancing effect of probucol reported previously, was not only confined to the aortic root region, but probucol also changed to become increasingly more anti-atherogenic the further distal the site examined (Table 1). At the abdominal aorta probucol effectively prevented lesion formation.

(b) Aortic Biochemistry

The contents of lipids and antioxidants in the entire aortas of control and probucol-treated mice were measured. Feeding apoE−/− mice a high fat diet for 24 weeks substantially increased the aortic content of lipoprotein-derived lipids, including cholesteryl linoleate (C18:2, the major readily oxidisable lipid) and α-tocopherol (vitamin E). Tables 2 & 3 show the values expressed per protein for the major lipids and antioxidants obtained after 24 weeks high fat diet. In addition to non-oxidized lipids, aortas also contained LOOH and CE-OH (Table 2) despite the presence of substantial amounts of the antioxidant vitamins E and C (Table 3). The presence of LOOH was confirmed by HPLC with post-chemiluminescence detection, with chemiluminescence positive signals being eliminated by borohydride treatment. Overall ˜1% of the aortic lipid was oxidised (Table 2), and aortas also contained ubiquinone-10 and α-tocopherylquinone, the oxidized forms of ubiquinol-10 and α-tocopherol, respectively (Table 3).

TABLE 2
Aortic and plasma lipids in apoE−/− mice after 24 weeks of intervention

| | Aorta (nmol/mgp) | | Plasma (mM) | |
|---|---|---|---|---|
| | Control | Probucol | Control | Probucol |
| Triglyceride | — | — | 0.8 ± 0.0 | 0.4 ± 0.0* |
| C | 969 ± 295 | 173 ± 60* | 6.7 ± 3.0 | 2.6 ± 1.2* |
| C20:4 | 20 ± 5 | 4.0 ± 2* | 0.3 ± 0.1 | 0.2 ± 0.2 |
| C18:2 | 64 ± 18 | 12 ± 6* | 3.0 ± 1.2 | 1.3 ± 0.8* |
| LOOH | 0.35 ± 0.16 | 0.080 | nd | nd |
| CE-OH | 0.69 ± 0.29 | 0.17 ± 0.08* | 0.4 '3 10⁻³ ± 0.3 × 10⁻³ | 0.2 × 10⁻³ ± 0.2 × 10⁻³ |

ApoE−/− mice were fed either a control or probucol-supplemented diet for 24 weeks before aortas and plasma were analysed.

TABLE 3
Aortic and plasma antioxidants in apoE−/− mice after 24 weeks of intervention

| | Aorta (pmol/mgp) | | Plasma (μM) | |
|---|---|---|---|---|
| | Control | Probucol | Control | Probucol |
| Tocopherol | 1097 ± 123 | 380 ± 138* | 39.4 ± 18.9 | 10.5 ± 3.50* |
| Tocopherylquinone | 183 ± 28 | 30 ± 2* | nd | nd |
| Ubiquinone-9 | 280 ± 78 | 114 ± 47* | 0.3 '5 0.1 | 0.3 ± 0.1 |
| Ubiquinol-10 | nd | nd | 0.02 ± 0.04 | 0.01 ± 0.02 |
| Ubiquinone-10 | 64 ± 17 | 25 ± 10* | 0.2 '5 0.0 | 0.1 ± 0.1 |
| Total coenzyme Q | 344 ± 87 | 139 ± 57* | 0.5 | 0.4 |
| Ascorbate | 31 ± 18 | 322 ± 320 | 94.1 ± 38.0 | 129.2 ± 36.2* |
| Probucol | — | 4127 ± 1977* | — | 136.4 ± 99.3* |
| Bisphenol | ‘3 | 133 ± 56* | — | 4.0 ± 0.6* |
| Diphenoquinone | — | 315 ± 76* | — | 8.1 ± 4.4* |

ApoE−/− mice were fed either a control or probucol-supplemented diet for 24 weeks before aortas and plasma were analysed.

Compared to controls, probucol significantly decreased the aortic content of lipids and lipid-soluble antioxidants expressed per protein (Tables 2 & 3). For example, the concentrations of C, vitamin E and total CoQ (ubiquinones plus ubiquinols) decreased 5.6-, 2.9- and 2.5-fold, respectively. This reduction in aortic lipids by probucol is consistent with its known lipid lowering activity and the histological results of the present study (Table 1, FIG. 1). Probucol also significantly decreased the aortic content of protein-standardised CE-OH and, where analysed, LOOH (Table 2). To assess whether this was the result of a lipid lowering or antioxidant activity of probucol, we expressed the aortic content of oxidised lipids per CE (Table 4). Probucol did not affect the content of lipid standardised oxidized lipids, indicating that the drug acted as a lipid lowering rather than antioxidant agent. However, probucol increased the aortic concentration of the water-soluble antioxidant ascorbate, for unknown reasons. Probucol was detected at ˜11-fold higher concentration than vitamin E, and ˜11% of drug was metabolised to bisphenol or diphenoquinone.

TABLE 4
Aortic content of lipid-standardised oxidised lipids in apoE−/− mice
after 24 weeks of intervention

| | Control | Probucol |
|---|---|---|
| LOOH/LH (× 10³) | 4.4 ± 2.1 | 4.4^{a} |
| CE-OH/LH (× 10³) | 8.2 ± 3.1 | 12.5 ± 6.7 |

ApoE−/− mice were fed either a control or probucol-supplemented diet for 24 weeks before aortas were analysed.

(c) Plasma Lipids

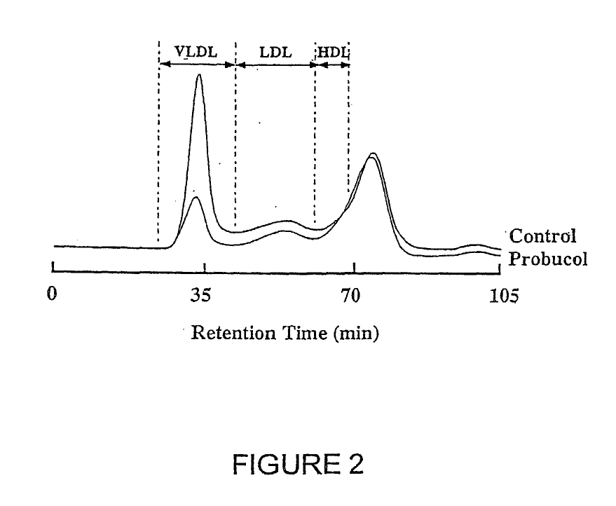

Plasma from probucol treated mice had significantly less lipid (Table 2) and vitamin E was decreased -4-fold, whereas ascorbate was increased ˜1.4-fold and total coenzyme Q remained unchanged (Table 3). Similar to the situation in the aorta, probucol was present at ˜13-fold higher concentration than vitamin E and ˜8% of the drug converted into bisphenol or diphenoquinone, suggesting that metabolism of probucol does not take place in the vessel wall. Compared to the aorta, plasma contained only small amounts of CE-OH and there was no difference between the two groups. Furthermore, LOOH and α-tocopherylquinone were absent (Table 3) indicating that lipoprotein oxidation in apoE−/− mice occurs within the vessel wall rather than the circulation. Size exclusion chromatography showed that the majority of the probucol-induced lipid lowering action was due to a decrease in VLDL, with LDL and HDL remaining largely unchanged (FIG. 2). Thus, the content of C in VLDL from the probucol group was decreased 25% of the control value, reflecting the situation in plasma (cf. Tables 2 & 5). HPLC analysis also showed that the lipid-soluble antioxidants and their metabolites were distributed more or less proportional to the cholesterol content of the lipoproteins (Table 5). This could explain why probucol lowered plasma vitamin E.

TABLE 5
Lipids and antioxidants present in lipoprotein fractions prepared
from plasma of apoE−/− mice after 24 weeks of intervention

| | | | | | |
|---|---|---|---|---|---|
| | | | | | Total |
| Lipoprotein fraction | C | C20:4 | C18:2 | -TOH | CoQ |

Control

| | | | | | |
|---|---|---|---|---|---|
| VLDL | 2.6 | 71 | 1007 | 31 | 0.2 |
| LDL | 0.9 | 28 | 330 | 3 | 0.2 |
| HDL | 0.15 | 21 | 113 | 3 | 0 |

Probucol

| | | | | | |
|---|---|---|---|---|---|
| VLDL | 0.65 | 17.5 | 340 | 0.56 | 0.1 |
| LDL | 0.96 | 50.4 | 388 | 7.8 | 0.4 |
| HDL | 0.12 | 24.0 | 531 | 1.4 | 0.1 |

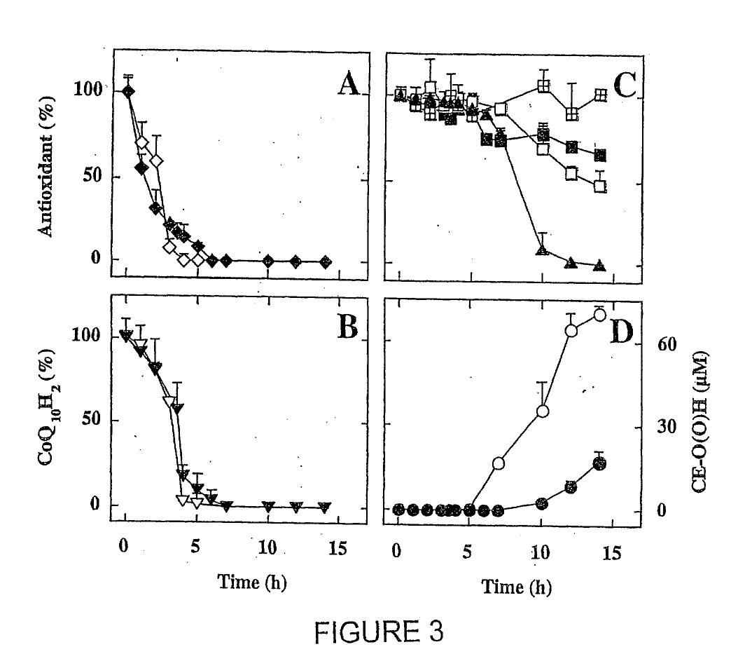

ApoE−/− mice were fed high fat diet without (control) or with 1% w/w probucol for 24 weeks before plasma was obtained, pooled and subjected to size exclusion FPLC. Two consecutive separations each employing 300 μL undiluted plasma were carried out. The corresponding eluents from both injections were collected, pooled according to fractions corresponding to very low-density lipoprotein (VLDL), LDL, and HDL plus mouse serum albumin (see FIG. 3) and then extracted and analysed for lipids and antioxidants.

(d) Plasma Lipoprotein Oxidizability

Enhanced resistance of plasma lipoproteins to oxidation is often used as a measure of antioxidant efficacy. Therefore, we examined AAPH-induced oxidation of pooled plasma from control and treated animals. Exposure of control plasma to this oxidant resulted in the time-dependent and concomitant consumption of ascorbate (FIG. 3A) and ubiquinols-9 and -10 (FIG. 3B). As expected from the increased starting concentration, the time required for ascorbate depletion was increased somewhat in the probucol group (FIG. 3A) and this was reflected in an increase in the time required for the complete consumption of ubiquinols (FIG. 3B). Upon depletion of ascorbate and ubiquinols, bisphenol (filled squares in FIG. 3C) was oxidised into diphenoquinone (not shown). Thereafter plasma vitamin E (squares in FIG. 3C) decreased, concomitant with the accumulation of CE-O(O)H (FIG. 3D), the onset and initial rate of which were delayed and decreased respectively, in plasma from probucol-treated mice (FIG. 3D) despite the four-fold lower concentration of vitamin E. Probucol (cross-hatched squares in FIG. 3C) remained unchanged throughout the oxidation period examined. These observations can be explained readily on the basis of tocopherol-mediated peroxidation. Together these data indicate that the increased concentration of ascorbate and the presence of the bisphenol rather than probucol afforded an enhanced resistance of plasma lipids to ex vivo oxidation induced by MPH.

In summary, the lipid-lowering antioxidant probucol can inhibit atherosclerosis in animals and restenosis in humans. However, probucol has been shown to promote atherosclerosis in the aortic root of apolipoprotein E-deficient deficient (apoE−/−) mice. Here we examined the effects of probucol on both lesion formation at four sites along the aorta and lipoprotein oxidation in plasma and aortas of apoE−/− mice receiving a diet containing 21.2% (wt/wt) fat and 0.15% (wt/wt) cholesterol without or with 1% (wt/wt) probucol. After 6 months, controls had developed lesions at all sites investigated. Lesion development was strongly (P=0.0001) affected by probucol, but this effect was not uniform: lesion size was increased in the aortic root but significantly decreased lesion in the aortic arch, the descending thoracic and proximal abdominal aorta. Plasma and aortas of probucol-treated mice contained high concentrations of probucol and its metabolites (bisphenol and diphenoquinone), increased vitamin C, markedly decreased VLDL (but not LDL and HDL) and decreased cholesterol, cholesterylesters, triglycerides, vitamin E and oxidised lipids compared to controls. Interestingly, probucol-treatment did not decrease the proportion of aortic lipids that were oxidised. Plasma vitamin C and bisphenol, but not probucol protected plasma lipids from ex vivo oxidation. These results show that like in other species, probucol can inhibit lesion formation in most parts of the aorta of apoE−/− mice. This effect may involve lipid oxidation-independent mechanisms localised within the vessel wall as well as lipid lowering.

### Example 2

Ability of Probucol to Inhibit Atherosclerosis/Restenosis in Cholesterol-Fed, Ballooned New Zealand White Rabbits Independent of Lipoprotein Oxidation in the Vessel Wall.

This example illustrates the dissociation of inhibition of LDL oxidation and ability of Probucol to inhibit atherosclerosis/restenosis.

Eighteen New Zealand white rabbits (15 weeks old) received a 2% (wt/wt) cholesterol fortified diet without or with 1% (wt/wt) probucol for 6 weeks. Aortic endothelial denudation was performed at week 3 by withdrawing an inflated 3F Fogarty balloon embolectomy catheter three times down the length of the aorta. Plasma was obtained from animals as described in Example 1 at the start and end of the study. At the end of the study period, individual aortas were removed and prepared for HPLC analysis as in Example 1. Lipids and antioxidants including probucol and its metabolites, bisphenol and diphenoquinone, were analyzed by RP-HPLC as described previously. Aortas designated for histology were perfusion-fixed with 4% (v/v) formaldehyde and transferred into formalin. Cross-sections (2-3 μm thick) were prepared and stained with Weigert's hematoxylin-van Gieson, and the intima to media ratio determined for three sections.

Significant concentrations of probucol and its metabolites were measured in plasma and aorta. Probucol significantly decreased the content of plasma and aortic lipids, and the absolute amounts of oxidized lipids compared with respective controls. However, the proportion of aortic lipids that was oxidized was not affected by probucol. Probucol significantly decreased atherosclerosis, demonstrating significant protective effects on atherosclerosis. The widely held view that probucol inhibits atherosclerosis by inhibiting lipid oxidation, a process thought to be central to atherogenesis, is not supported by these findings.

### Example 3

Ability of Probucol to Promote Re-Endothelialization in Cholesterol-Fed and Normal Chow Fed, Ballooned New Zealand White Rabbits.

This example illustrates that of probucol is able to promote re-endothelialization in vivo and that the ability of probucol to promote re-endothelialization in vivo is independent of its lipid-lowering activity. The results are shown in table 6 below:

TABLE 6
Reendothelialization in cholesterol-fed and normal chow fed ballooned
rabbits

Re-endothelialized
area (% total surface area)

| | Thoracic Aorta | Abdominal Aorta |
|---|---|---|
| 2% Cholesterol-fed | | |
| Control | 45.6 ± 4.5 | 44.2 ± 5.6 |
| Probucol | 57.3 ± 5.1* | 55.1 ± 7.1** |
| Normal Chow | | |
| Control | 37.6 ± 6.7 | 35.2 ± 5.5 |
| Probucol | 50.4 ± 6.4** | 48.2 ± 9.0*** |

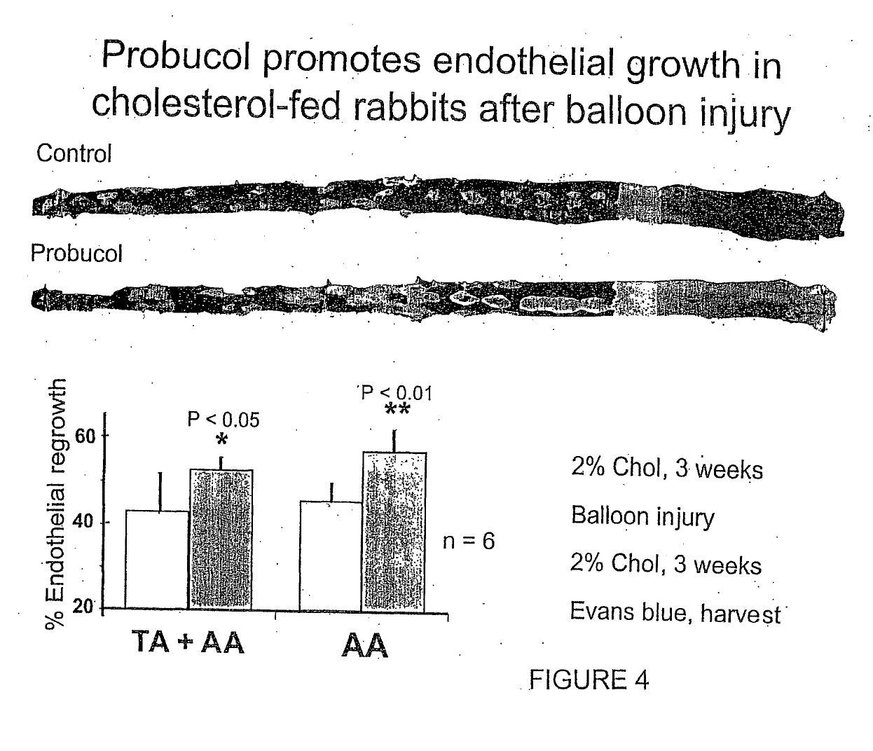

Results are from two separate experiments and represent mean±SD of six (cholesterol diet) and five animals (normal diet) per group. Asterisk indicates statistically significant higher values compared with corresponding controls, with p-values of 0.002, 0.015 and 0.025 for *, ** and ***, respectively. FIG. 4 also illustrates the re-endothelialization in the control and probucol arteries.

### Example 4

Ability of a Co-Antioxidant (Bisphenol) to Inhibit Lipoprotein Oxidation in the Vessel Wall of Animals.

This example illustrates that co-antioxidants can inhibit in vivo lipid oxidation.

(a) Animals

Male apoE−/−;LDLr−/− mice (59 total), were maintained on standard R3 chow from weaning to 8 weeks of age (young controls, n=9). Thereafter mice received R3 chow with (n=25) or without supplemented bisphenol (n=25) for an additional 14 weeks. Standard R3-mouse chow was fortified with bisphenol at 0.03% (w/w), a level of supplementation shown in pilot experiments to afford circulating concentrations of the drug of ˜200 μM, considered suitable to test the effect of the co-antioxidant on atherogenesis in this animal model. At 22 weeks of age 10 and 15 mice of each group were used for biochemical and histological analyses, respectively. Due to the small size, it was necessary to pool aortas to yield sufficient material for biochemical analyses.

(b) Blood Sampling and Preparation of Plasma and Serum.

Blood samples from control and drug-treated apoE−/−;LDLr−/− mice (˜1 mL) were taken by direct cardiac puncture.

(c) Perfusion and Fixation of Aortic Vessels.

Mouse aortas were excised as follows: after bleeding, the heart was perfused with Dulbecco's phosphate-buffered saline containing 100 μM BHT and 1 mM EDTA (maximum pressure 80 mm Hg) through the left ventricle, the right side chamber being opened to allow flow. For histological samples only, the vasculature was subsequently fixed with formal saline. The hearts and entire aortas from all treatment groups were removed and immediately cleaned of fat and connective tissue. Aortas for biochemical analyses were frozen immediately (−70° C.) without formalin fixation.

(d) Evaluation of Atherosclerosis.

Aortic lesions were assessed in segments centered around the third pair of intercostal artery branches in the descending thoracic aorta. Briefly, the fixed aortas were dehydrated in ethanol, cleared with xylene and embedded in paraffin. Serial sections (10 in total; each 2-3 μm thick and 100 μm apart) were cut and stained using Weigerts hematoxylin-van Gieson. Aortic thickening was assessed as the total volume of intima in the segment investigated in bisphenol-treated versus control samples.

Aortic volumes were determined by planimetry, using a Lucivid device (MicroBrightField, Colchester, Vermont, Canada) attached to a Leitz DRM microscope that allowed the superposition of a computer monitor onto the cross sectional image.

(e) Preparation of Aortic Homogenates.

Cleaned aortic segments were thawed, blotted, pooled (n=7 or 8), weighed, added to 2 mL of argon-flushed phosphate-buffered saline (to give ˜40 mg wet tissue/mL) containing BHT (100 μM) and EDTA (1 μM). The tissue was minced with scissors, isoascorbate (5 μM) and α-tocotrienol (1 μM) added as internal standards for ascorbate and vitamin E (including α-TQ), respectively, and the samples transferred to a polytetrafluoroethylene-lined glass tube and homogenised at 4° C. for 5 minutes using a teflon piston rotating at 500 r.p.m. For recovery of oxidised lipids, [³H]-Ch18:2-OH was incorporated into human LDL and added to the vessel prior to homogenisation. Analysis of spiked homogenate showed 94±1.3% recovery of the label (mean±range for 2 separate experiments). For ascorbate measurement, raw homogenate (50 μL) was added to metaphosphoric acid (5% v/v, 50 μL) and frozen on dry ice. Immediately before HPLC analysis, the aliquots were thawed and diluted with phosphate buffer (50 μL, 250 mM, pH 7.4), to adjust the pH. For the analyses of lipids, the remaining homogenate (−1.8 mL) was divided into 4×450 μl aliquots and each extracted with chilled methanol (2 mL) and hexane (10 mL). Hexane phases were combined and evaporated to dryness and the residue resuspended in isopropanol (200 μL).

(f) Oxidation of Mouse Plasma.

Oxidation of plasma, pooled from=3 mice was carried out by addition of the peroxyl radical generator (final concentrations 5 mM) and incubating the reaction mixture at 37° C. under air. Aliquots (50 μL) of the reaction mixture were removed, extracted in methanol/hexane (1:5, v/v), and the consumption of antioxidants and accumulation of lipid oxidation products determined.

(g) Analysis of Lipid and Water-Soluble Compounds.

Analyses of oxidised and non-oxidised lipids were carried out by RP-HPLC as described in Example 1 except that in some instances UV₂₃₄ nm rather than post-column chemiluminescence detection was used to measure C18:2-OOH and the corresponding hydroxides (together referred to as CE-O(O)H) which show similar retention times under these chromatographic conditions. α-TQ, α-TOH, α-tocotrienol, D-isosascorbate and ascorbate were determined by HPLC with electrochemical detection. For oxidation of plasma, unesterified cholesterol (which remained non-oxidised in these experiments) was employed as internal standard for all polyunsaturated, lipid-soluble components analysed. Bisphenol and diphenoquinone were analysed by RP-HPLC: flow 1.5 mL/min, 100% solvent A (MeCN/MeOH/H₂O 10:10:3, v/v/v) for 0-15 min monitored at 270 nm, followed by 50% solvent A and B (MeCN/MeOH 1:1, v/v) for 15-22 min at 242 nm and then 100% B for 22-28 min at 420 nm. Bisphenol and diphenoquinone eluted at 9 and 27 min, respectively. All compounds were quantified by peak area comparison with authentic standards. Where indicated, total cholesterol and triglyceride were assayed enzymatically (Boehringer, Mannheim, Germany).

ApoE−/−;LDLr−/− mice readily develop detectable lesions after only 15-weeks of standard chow diet, indicating that these animals are suitable to study both early events in atherogenesis and its inhibition. Table 7 summarises the plasma levels of lipids, α-TOH and bisphenol in mice receiving a control or bisphenol-fortified diet. There was an age-dependent increase in plasma total cholesterol, triglycerides and vitamin E as judged by comparing data from 8 and 22 weeks old control animals. After 14 weeks intervention, plasma levels of bisphenol plus diphenoquinone reached 216 μM. Supplementation of the diet with bisphenol significantly increased total plasma cholesterol, whereas triglycerides were unchanged and vitamin E levels decreased significantly compared to age-matched controls (Table 7).

TABLE 7
Plasma lipid, vitamin E and drug levels in apoE−/−; LDLr−/− mice.^{‡}

| | | | | Total |
|---|---|---|---|---|
| | Total Cholesterol | Triglycerides | Vitamin E | Drug |
| Group | (mm) | (mm) | (μm) | Level |
| Young | 13.2 (0.4) | 1.6 (0.2) | 21.7 (0.7) | — |
| Control | | | | |
| Age | 22.6 (2.4) | 5.2 (0.9) | 36.6 (3.1) | — |
| matched | | | | |
| Control | | | | |
| Bisphenol | 25.2* (1.2) | 6.3 (0.5) | 22.9* (0.8) | 216 (25) |

Animals were fed Lactamin R3-chow containing either 0.03% bisphenol or vehicle alone to act as control. Young and age-matched control animals were sacrificed at 8 and 15 weeks of age, respectively.

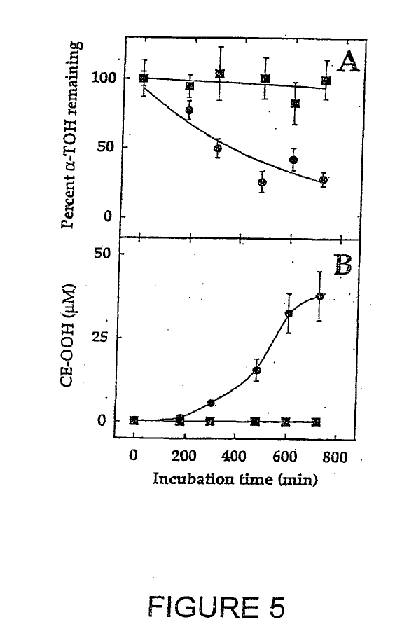

Samples of pooled plasma from bisphenol-treated mice were markedly resistant to peroxyl radical-induced ex vivo lipid peroxidation compared with age-matched controls (FIG. 5). Thus, even after 12 h of oxidation at 37° C., α-TOH remained unaltered (FIG. 5A) with <1 μM CE-OOH detected. By contrast, ^{˜}70% of α-TOH was consumed and >30 μM CE-OOH accumulated in the corresponding control plasma (FIG. 5B), fully consistent with plasma lipid peroxidation proceeding via tocopherol-mediate peroxidation (TMP). Separate studies showed that this resistance to peroxyl radial-induced ex vivo lipid peroxidation was directly attributable to bisphenol, as the bisphenol rather than α-TOH was consumed during the period of oxidation. The corresponding oxidation product, diphenoquinone, is incapable of acting as an co-antioxidant, as judged by its high anti-TMP index and inability to cause the decay of α-tocopheroxyl radical (Table 8).

TABLE 8
Anti-TMP and tocopheroxyl radical attenuating ability (TRAA) indices
for bisphenol, diphenoquinone and BHT.

| Compound | Structure | Anti-TMP index* | TRAA‡ |
|---|---|---|---|
| H 212/43 | 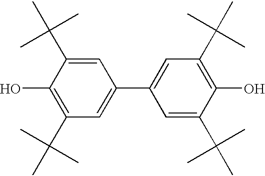 | 3.2 | Immediate decay |
| H 330/68 | 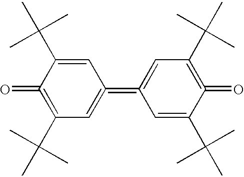 | 100 | k_{obs(+)}/k_{obs(-)} ˜ 1 |
| BHT | 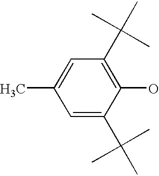 | 8-10 | Immediate decay |

*Determination of the Anti-TMP index has been described previously (J. Biol.Chem 1995;270:5756-5763).
‡Assessment of tocopheroxyl radical attenuating ability (TRAA) was performed as described in J. Lipid Res 1996;37:853-867; k_{obs} is the observed rate constant measured for the decay of -tocopheroxyl radical in the presence (+) or absence (−) of the test compound.

For biochemical analyses it was necessary to pool aortas to yield sufficient material to detect the various lipids and antioxidants, despite the use of HPLC with sensitive detection. As a result of this limitation, tissue parameters were determined as the mean of duplicate analyses on two separate pools of aortas of each the control and bisphenol-treated mice. The results (Table 9) show that concentrations of ascorbate, unesterified cholesterol and CE in aortic homogenates were similar in the two treatment groups, although a marginal decrease in CE was seen in a sub-group of drug-treated mice with high plasma levels of bisphenol. By contrast, the levels of aortic A-TOH were lower in drug-treated than age-matched controls.

TABLE 9
Aqueous and lipophilic parameters of apoE−/−; LDLr−/− mouse aortae.^{‡}

| | | | | Bisphenol |
|---|---|---|---|---|
| | Control 1 | Control 2 | Bisphenol [low]^{†} | [high]^{†} |
| Ascorbate | 2662 | 2606 | 3204 | 2570 |
| C^{§} | 226 | 204 | 204 | 190 |
| C20:4^{§} | 10.6 | 12.0 | 12.7 | 8.5 |
| C18:2^{§} | 19.7 | 19.7 | 21.0 | 13.3 |
| CE-O(O)H | 112.7 | 140.8 | 14.1 | 2.8 |
| CE-O(O)H/LH^{¶} | 1.4 | 1.6 | 0.145 | 0.002 |
| α-TOH | 1239 | 1070 | 669 | 586 |
| α-TQ | 12.0 | 13.4 | 4.9 | 3.5 |

Unless otherwise stated lipid and antioxidant levels are expressed as pmol/mg protein. LH, lipid containing bisallylic hydrogens. §Values are expressed as nmol/mg protein. Protein ranged from 2.4-3.3 mg/mL. ¶Value given represents ratio×10³. †Mice were ranked in order of plasma concentration of bisphenol, then corresponding aortae were pooled into groups of 7 and 8 on the basis of plasma drug-levels giving two distinct groups. Low and high bisphenol correspond to 106±40 and 212±50 μM, respectively. Control aortas were pooled randomly into two groups of 7 and 8 respectively.

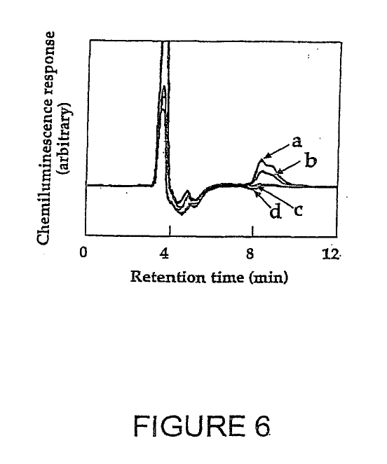

Importantly, aortic tissue of control mice contained significant amounts of oxidised lipids, with approximately 0.15% of the CE present as CE-O(O)H (Table 9). Strikingly, the level of these oxidised lipids were 10- and 1000-fold lower in aortas from drug-treated animals with low and high plasma levels of bisphenol, respectively, particularly when expressed per percent lipid (Table 8). FIG. 6 shows representative traces of HPLC with post-column chemiluminescence detection. CE-OOH, detected in the organic extracts of aortas of control but not bisphenol-treated mice, eluted between 8 and 10 minutes. Treatment of the control samples with sodium borohydride eliminated these chemiluminescence-positive peaks (not shown), indicating their nature as hydroperoxides. α-TQ, an additional marker of biological lipid oxidation, was also decreased in aortas of bisphenol vs control mice (Table 8), although the extent of this inhibition was much less than that observed for CE. Linear regression analyses indicated that there were no significant correlations (R<±0.5) between any of the plasma and tissue parameters measured.

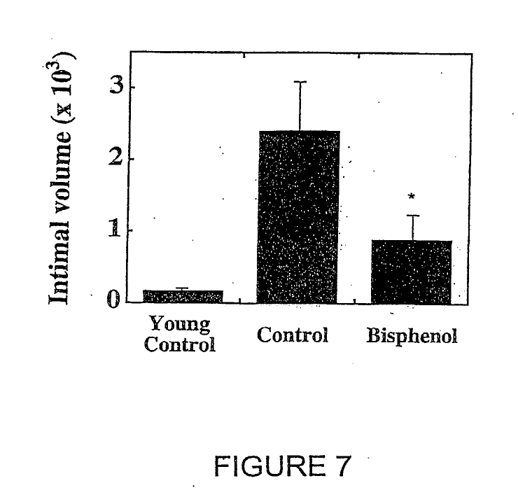

The intimal volume in the descending thoracic aortas of control apoE−/−;LDLr−/− mice fed the standard chow increased more than 10-fold from 8 to 22 weeks of age (FIG. 7). Administration of bisphenol for 14 weeks substantially decreased the lesion size, as judged by a significant decrease in aortic volume compared with age-matched controls, although the intimal volume in the drug-treated (older) animals remained higher than that determined for young control mice (FIG. 7).

In summary, antioxidants can inhibit atherosclerosis in animals, though it is not clear if this is due to the inhibition of aortic lipoprotein lipid oxidation. Co-antioxidants inhibit radical-induced, tocopherol-mediated peroxidation of lipids in lipoproteins through elimination of tocopheroxyl radical. Here we tested the effect of the bisphenolic probucol metabolite and co-antioxidant bisphenol on atherogenesis in apoE−/−;LDLr−/− mice, and how this related to aortic lipid oxidation measured by specific HPLC. Dietary supplementation with bisphenol resulted in circulating drug levels of ˜200 μM, increased plasma total cholesterol slightly and decreased plasma and aortic α-tocopherol significantly relative to age-matched control mice. Treatment with bisphenol increased the antioxidant capacity of plasma, as indicated by prolonged inhibition of peroxyl radical-induced, ex vivo lipid peroxidation. Aortic tissue from control apoE−/−;LDLr−/− mice contained lipid hydro(pero)xides and substantial atherosclerotic lesions, both of which were decreased strongly by supplementation of the animals with bisphenol. The results show that a co-antioxidant effectively inhibits in vivo lipid peroxidation and atherosclerosis in apoE−/−;LDLr−/− mice, consistent with though not proving a causal relationship between aortic lipoprotein lipid oxidation and atherosclerosis in this model of the disease.

### Example 5

Inhibition of Restenosis by probucol Dithio Analogue

To examine the potential of the probucol dithio analogue (III), having the molecular weight of 474, (PDA474) to inhibit restenosis, we tested its effect on intimal thickening in rabbit aorta after arterial balloon injury (ABI). PDA474 was synthesised and supplied in pure form (99%) by Polysciences, Inc. (400 Valley Road, Warrington, Pa. 18976, USA).

Twelve New Zealand White rabbits were fed a standard rabbit chow (100 g per day) with (n=6) or without (n=6) 0.02 weight % PDA474 (in dry powder form) for 9 weeks. The initial dosage was based on in vitro studies and other useful dosage amounts can be easily derived using a similar approach.

At the end of week 3, the abdominal aorta of all rabbits was injured by ABI. At the end of week 9, the infra-renal abdominal aorta was removed, fixed in 4% buffered formalin, embedded in paraffin, sectioned and stained with Verhoeff Hematoxylin. The intima and media ratio (IMR) was then determined by tracing the intimal and medial areas, and dividing their respective total pixel numbers. Higher IMR indicate larger lesions.

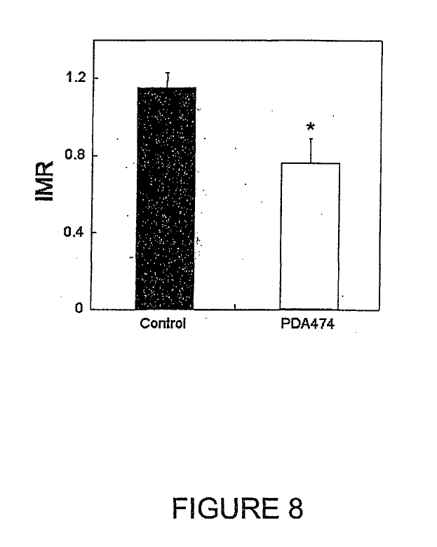

One control rabbit collapsed after ABI surgery and another control rabbit died after ABI from urinary infection, decreased the number of control rabbits to four. All of the drug-treated animals survived and were included in the analyses and data presentation. As expected, ABI followed by six weeks of normal chow feeding resulted in substantial intimal thickening of the infra-renal abdominal aorta. Treatment of rabbits with 0.02% PDA474 (open bar in FIG. 8) resulted in a significantly (p<0.05) lower IMR (i.e., 0.76±0.13, mean±SEM, n=6) when compared to control rabbits (empty bar, 1.15±0.08 (n=4). The 34% lower IMR in the drug-treated animals supports the idea that PDA474 inhibits restenosis.

This example demonstrates that a dithio analogue of probucol, such as that exemplified by PDA474, is effective in inhibiting restenosis.

Finally, it is understood that various modifications, alterations and/or additions may be made to the example specifically described and illustrated herein without departing from the spirit and scope of the invention.

The claims defining the invention are as follows:

1. Composition comprising an effective promoter of re-endothelialization and an effective inhibitor of lipoprotein oxidation.

2. A composition according to claim 1, wherein the promoter of re-endothelialization is probucol or an analogue thereof.

3. A composition according to claim 1, wherein the inhibitor of lipoprotein oxidation is a co-antioxidant.

4. A composition according to claim 3, wherein the co-antioxidant is probucol-derived bisphenol.

5. A composition according to claim 4, wherein said probucol-derived bisphenol has the following structure:

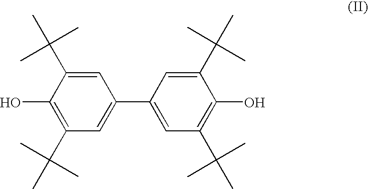

6. Composition comprising a compound that possesses both re-endothelialization-promoting and lipoprotein oxidation-inhibitory activity.

7. A composition according to claim 6, wherein said compound is a probucol analogue having modifications in the region of the ‘central bridge’ that allows intra-cellular reduction to a mercaptophenol.

8. A compound according to claim 7, wherein the analogue has the following, or related, structure:

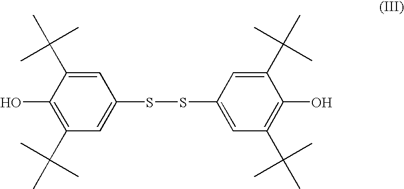

9. A compound according to claim 8, wherein the analogue is PDA474.

10. A pharmaceutical composition comprising the compound according to claim 8 or claim 9.

11. A pharmaceutical composition according to claim 10, further comprising one or more effective inhibitors of lipoprotein oxidation.

12. A pharmaceutical composition according to claim 11, wherein the inhibitor of lipoprotein oxidation is a co-antioxidant.

13. Method for prophylactic or therapeutic treatment of cardiovascular diseases, said method comprising the administration to a subject requiring such treatment an effective promoter of re-endothelialization and an effective inhibitor of lipoprotein oxidation.

14. A method according to claim 13, wherein the effective promoter of re-endothelialization and the effective inhibitor of lipoprotein oxidation are administered simultaneously.

15. A method according to claim 13, wherein the effective promoter of re-endothelialization and the effective inhibitor of lipoprotein oxidation are administered sequentially in any order.

16. A method according to claim 13, wherein the promoter of re-endothelialization is probucol or an analogue thereof.

17. A method according to claim 13, wherein the treatment promotes re-endothelialization of damaged vessel walls in vivo.

18. A method according to claim 17, wherein the subject is administered probucol and probucol-derived bisphenol.

19. Method for the prophylactic or therapeutic treatment of cardiovascular diseases, said method comprising the administration to a subject requiring such treatment a compound that possesses both re-endothelialization-promoting and effective lipoprotein oxidation-inhibitory activity.

20. A method according to claim 19, wherein the compound has the following, or related, structure:

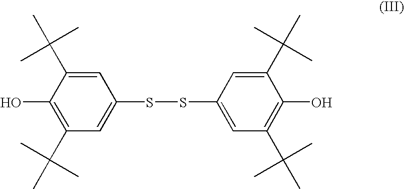

21. A method according to claim 20, wherein the compound is PDA474.

22. A method according to claim 20 or claim 21, wherein the compound is administered in conjunction with one or more other compounds which have lipoprotein oxidation-inhibitory activity.

23. A method according to claim 22, wherein the compound which has lipoprotein oxidation-inhibitory activity is a co-antixodant.

24. A method according to claim 23, wherein the co-antioxidant is probucol-derived bisphenol.

25. Method of treatment which promotes the re-endothelialization of damaged vessel walls in vivo, said method comprising administering to a subject requiring such treatment an effective amount of a promoter of re-endothelialization and an effective inhibitor of lipoprotein oxidation.

26. A method according to claim 25, wherein the promoter of re-endothelialization is probucol or an analogue thereof.

27. A method according to claim 26, wherein the promoter of re-endothelialization and the effective inhibitor of lipoprotein oxidation are a compound having the following, or related, structure:

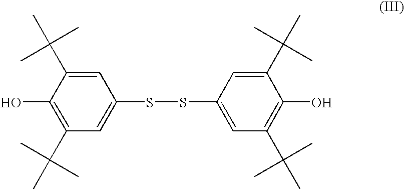

28. A method according to claim 27, wherein the compound is PDA474.

29. Method for the prophylactic or therapeutic treatment of cardiovascular diseases, said method comprising administering to a subject requiring such treatment an effective amount of probucol or an analogue thereof, to promote re-endothelialization.

30. Re-endothelialization composition comprising probucol or an analogue thereof and a pharmaceutically accepted carrier.

31. Re-endothelialization composition comprising a compound having the following, or related, structure:

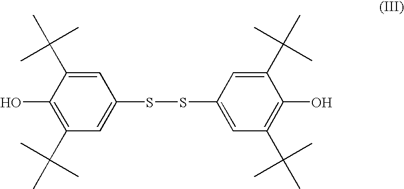

32. Re-endothelialization composition according to claim 31, wherein the compound is PDA474.

33. Re-endothelialization composition according to any one of claims 30 to 32, further comprising one or more effective inhibitors of lipoprotein oxidation.
